(19) United States
(12) Patent Application Publication
(10) Pub. No.: US 2024/0425815 A1
(43) Pub. Date: Dec. 26, 2024

(54) READY-TO-USE STEM CELL THERAPEUTIC

(71) Applicant: AARHUS UNIVERSITET, Aarhus C (DK)

(72) Inventors: Hermanus Johannes Marco EIJKEN, Aarhus C (DK); Toke ALSTRUP, Aarhus C (DK)

(21) Appl. No.: 18/691,971

(22) PCT Filed: Sep. 21, 2022
(86) PCT No.: PCT/EP2022/076148

§ 371 Date: Mar. 14, 2024

(30) Foreign Application Priority Data
Sep. 23, 2021 (EP) 21198531.2

(51) Int. Cl. C12N 5/0775; A01N 1/02; A61K 35/28; C12N 13/00

(52) U.S. Cl. CPC C12N 5/0667; A61K 35/28; C12N 13/00; A01N 1/0284; C12N 2523/00

(57) ABSTRACT

The present invention relates to inactivated stem cells, which can be stored for several days in a refrigerator (above 0° C.), preferably in the range 0.1-10° C.) and still maintain their cellular and phenotypic integrity as well as therapeutic potential. In particular, the present invention relates to medical uses of such refrigerator-stored inactivated stem cell solution.

### TECHNICAL FIELD OF THE INVENTION

The present invention relates to inactivated stem cells, which can be stored for several days in a refrigerator (above 0° C., preferably in the range 0.1-10° C.) and still maintain their cellular integrity and therapeutic potential. In particular, the present invention relates to medical uses of such refrigerator-stored inactivated stem cell solution.

### BACKGROUND OF THE INVENTION

Cellular therapy such as MSC therapy is a promising field in medicine. The combination of MSC's regenerative and immunomodulatory properties has triggered exploration of the therapeutic application of MSCs for tissue regeneration and immunomodulation [1]. Currently multiple clinical phase II and III trials are ongoing for a wide variety of indications such as graft-versus-host disease, ischemic cardiac disease, knee osteoarthritis, organ transplantation and critical limb ischemia [1, 2].

At least two theories exist in relation to how MSCs may work in MSC therapy. One theory is that administered MSCs migrate to sites of injury, engraft, and differentiate into other cell types. Another theory is that the positive effects of MSCs are mainly mediated by the host cells, which are actively and passively activated by the administered MSCs [1, 3]. In a passive role, MSC may be phagocytosed by innate immune cells that subsequently adapt a more immunoregulatory and regenerative phenotype [4].

Although initial MSC clinical trials used autologous MSCs, the practical advantages of allogeneic MSC use are favouring these as candidates for stem cell-based therapies. The use of pre-banked allogeneic MSC is now considered the only feasible strategy for acute treatments and might be the only economically sustainable alternative for the majority of indications.

The use of pre-banked stem cells comes with a big disadvantage. After production, the product needs to be stored at cryogenic temperatures and shipped at these very low temperatures under continuously monitoring. Moreover, cryogenic storage facilities are needed in close proximity of the point of care. Cryopreserved products contain toxic compounds needed for cryo-storage, which is highly unwanted in case of local treatments. Hence, in order to administer MSCs, the staff needs to manipulate the product by thawing and formulation of the MSC product to a balanced salt solution. This process not only requires an expensive clean room laboratory infrastructure, which is not present at the majority of clinical institutes, it also nullify the quality controls and potency assays performed by the manufacturer before shipment. These factors greatly reduce applicability for many indications as well as increasing cost of therapy.

Intriguingly in some animal disease models it appears that dead or apoptotic MSCs show similar or even better therapeutic efficacy as their living counterparts [3, 5]. After inactivation by methods such as heat inactivation, MSCs become metabolically inactive and are unable to proliferate, to adhere and to secrete factors. Despite the metabolic inactive nature of these cells, heat inactivated MSCs (HI-MSCs) still maintain their immunophenotype. In a mouse sepsis model, i.v. administration of HI-MSCs was able to dampen inflammation and resulted in better overall survival compared to normal MSCs [6, 7]. In-vitro HI-MSC are able to modulate monocyte function and polarization, but are unable to modulate T-cell proliferation or regulatory B-cell formation [6].

It is standard practice in the field that HI-MSCs are directly used after heat inactivation (within hours after heat inactivation) [7].

Richard Burt et al discloses that mitotically inactivated embryonic stem cells can be used as an in vivo feeder layer to nurse damaged myocardium after acute myocardial infarction (Circ Res. 2012 Oct. 26; 111(10):1286-96). In Burt et al cells are inactivated with γ irradiation (40 Gy) and cryopreserved.

WO 2008/011524 A2 (Richard Burt) is directed to the use of mitotically inactivated stem cells for the repair of damaged organs and/or tissues.

Luk et al discloses that heat-inactivated mesenchymal stem cells (MSC) are unable to respond to inflammatory signals or secrete immunomodulatory factors, but preserve their cellular integrity [6] (STEM CELLS AND DEVELOPMENT. Volume 25, Number 18, 2016).

Hence, an improved method of storing inactivated MSCs would be advantageous, and in particular a more efficient and/or reliable method of using inactivated MSCs would be advantageous.

### SUMMARY OF THE INVENTION

In here is presented the novel finding that (heat) inactivation of MSCs stabilizes the structural integrity whilst maintaining the phenotype of MSCs (see e.g. examples 2-4). This surprisingly allows storage of these inactivated MSC's in e.g. a non-cytotoxic balanced salt solution at refrigerator temperature (ca. 2-8°), ready to be administered to a subject in need thereof.

Further the example section discloses:

Example 7: Shows that StemShells are cleared within 2 days when applied locally in-vivo, while still being able to induce an immunosuppressive phenotypic shift (iNOS⁻ and CD206⁺) of macrophages in vivo.

Example 8: Shows that bone marrow derived MSCs can also be stored for longer periods at refrigerator temperatures and at room temperature after they have been inactivated by heat inactivation. Thereby documenting that the method of the invention is generally applicable to different types of MSCs.

Example 9: Shows that StemShells can be produced from cryopreserved MSCs directly after thawing.

Example 10: Shows that heat inactivation to produce StemShells can be performed in protein free solutions such as saline or PBS, solutions supplemented with protein such human serum albumin and complex mixtures such as cell culture medium.

More specifically, the present invention is based on the discovery that inactivated stem cells can be stored for several weeks in a refrigerator (above 0° C., preferably in the range 0.1-10° C.) while maintaining their cellular integrity and therapeutic potential. Thus, the general assumption in the field that inactivated stem cells should be instantly cryo-preserved or instantly used, appears not to be correct.

This realization has several advantages in a medical setting. Advantages of the process and provided cells according to the present invention are but not limited to:
The allowance of storage at temperatures above 0° C. makes transportation cheaper and easier.
The recipient location does not need to have cryo-storage facilities.
The inactivated cells can be stored in a ready-for-use setting, such as in a syringe or vial/container formulated in a ready to use injection solution.

Thus, an object of the present invention relates to the provision of an improved method of storing inactivated stem cells.

In particular, it is an object of the present invention to provide an improved storage method that solves the above-mentioned problems of the prior art with expensive cryo-preservation facilities allowing for easy transportation between the site of stem cell inactivation and the site where the inactivated stem are to be used as medicaments in different treatment protocols.

It is also an object of the present invention to provide novel inactivated stem cells. In the present context, the inactivated stem cells according to the invention may be termed “StemShells”. The inactivated stem cells (or “StemShells”) may be used as medicaments, such as in the treatment or alleviation of diseases treatable with traditional stem cells or cryo-preserved stem cells.

Thus, one aspect of the invention relates to a process for providing mitotically and metabolically inactivated mesenchymal stem cells (MSCs), the process comprising
a) mitotically and metabolically inactivating MSCs provided from a subject;
b) storing the inactivated cells at a temperature in the range 0.1-25° C., preferably 0.1-10° C., for a period of at least 2 days, such as at least 5 days, such as at least 7 days, such as at least 14 days, such as at least 45 days; and
c) providing inactivated MSCs, such as inactivated cells being ready for use as a medicament.

Another aspect of the present invention relates to metabolically inactivated mesenchymal stem cells (MSCs) obtained/obtainable by a process according to the invention.

Yet another aspect of the present invention is to provide metabolically inactivated mesenchymal stem cells (MSCs) according to the invention, for use as a medicament.

Still another aspect of the present invention is to provide metabolically inactivated mesenchymal stem cells (MSCs) for use as a medicament, wherein said MSCs have been stored at a temperature in the range 0.1-10° C., for a period of at least 2 days, such as at least 5 days, such as at least 7 days, such as at least 14 days, such as at least 45 days before use as a medicament.

Yet an aspect relates to a container or vial comprising the cells according to the invention. Preferably, the container or vial comprises the cells in a pharmaceutical acceptable medium.

In yet another aspect the invention relates to the in vitro use of metabolically inactivated mesenchymal stem cells (MSCs) according to the invention.

### BRIEF DESCRIPTION OF THE FIGURES

FIG. 1

StemShells stay structurally stable for multiple weeks when formulated in a protein free solution such as PBS. StemShells were reconstituted and stored in (A) MEM medium supplemented with albumin or (B) in PBS. After 2 months of storage at refrigerator temperatures, microscopic pictures (100×) were made. (C) StemShells concentration was quantified by flowcytometry after 2 days and 2 months of storage at refrigerator temperatures.

FIG. 2

StemShells stay structurally stable for multiple weeks in a clinical relevant solution such as saline. StemShells were reconstituted in saline and stored for up to 45 days at refrigerator temperatures. (A) Forwards scatter (FSC) and sidewards scatter (SSC) of StemShells during storage measured by flowcytometry. (B) Concentration of StemShells during storage measured by flowcytometry. (C) Concentration of StemShells (in saline solution) after 45 days of storage at refrigerator temperatures followed by 7 days storage at room temperature.

FIG. 3

Levels of MSC cell surface markers on StemShells during storage in saline at refrigerator temperatures. The median fluorescent intensities (MFI) of (A) CD73, (B) CD90, (C) CD44 and (D) CD105 were quantified using flowcytometry.

FIG. 4

Fresh StemShells and 14 days stored StemShells are phagocytosed by monocytes in a similar extent as fresh living MSCs. (A-B) Data for fresh living MSCs and fresh StemShells. (A) Amount of phagocytose demonstrated by the percent of monocytes that are positive for PKH26. (B) Amount of phagocytose demonstrated by the amount of PKH26 (median fluorescent intensity) present in the PKH26 positive monocyte population. (C-D) Data for 14 days stored StemShells (in saline in refrigerator). (C) Amount of phagocytose demonstrated by the percent of monocytes that are positive for PKH26. (D) Amount of phagocytose demonstrated by the amount of PKH26 (median fluorescent intensity) present in the PKH26 positive monocyte population.

FIG. 5

StemShells stored in saline for 45 days at refrigerator temperatures induce a CD163 upregulation on monocytes when co-cultured for 24 hours. Compared to controls StemShells increased CD163 expression on CD14+, CD16+ monocytes for all tested blood samples (derived from different individuals). Compared to living MSCs, StemShells were more potent in increasing CD163 expression on CD14+, CD16+ monocytes.

FIG. 6

FIG. 6A shows that StemShells are cleared from fat grafts within 2 days of transplantation which is in contrasted to their living counterparts (allogeneic MSCs which were not heat inactivated) which remained present for at least 5 days. FIG. 6B shows that macrophages become positive for PKH26 at day 2 indicating active phagocytosis and clearance of the StemShells. FIG. 6C shows that the number of pro-inflammatory macrophages (iNOS⁺) are reduced at day 2 and 5 in fat grafts supplemented with StemShells when compared to their living counterpart allogeneic MSCs. FIG. 6D shows that the anti-inflammatory subtype of macrophages (CD206⁺) is significantly increased at day 2 and 5 in fat grafts receiving StemShells when compared to their living counterpart allogeneic MSCs.

FIG. 7

FIG. 7 shows that bone marrow derived StemShells are structurally and phenotypically stable for at least 13 days when stored at 4° C. in a clinically relevant and directly injectable solution (isotonic saline 2% HSA). (A) StemShell concentration. (B) CD73 levels; (C) CD90 levels; (D) CD105 levels. (E) Shows that exposure of temperatures such as room temperature (RT) are well tolerated by StemShells derived from bone-marrow derived MSCs. (F) StemShells (from bone marrow derived MSCs) stored at 4° C. for 13 days+4 days at RT (microscopic picture, 40×).

FIG. 8

FIG. 8 shows that cryopreserved mesenchymal stem cells can be heat inactivated directly after thawing to produce StemShells. Structural stability is determined by (A) StemShell concentration; and phenotypic characteristics: (B) CD29 levels; (C) CD44 levels; (D) CD73 levels: (E) CD90 levels; and (F) CD105 levels.

FIG. 9

FIG. 9 shows that mesenchymal stem cells can be heat inactivated in different solutions, including solutions without protein additives, to produce StemShells which are structurally stable for at least 7 days when stored at 4° C. (A) StemShell concentration; and phenotypic characteristics: (B) CD29 levels; (C) CD44 levels; (D) CD90v: (E) CD73 levels; and (F) CD105 levels. Concentration and phenotypic characteristics were similar between the conditions where StemShells were generated by heat inactivated in different solutions.

### DETAILED DESCRIPTION OF THE INVENTION

### Definitions

Prior to discussing the present invention in further details, the following terms and conventions will first be defined:

### Mesenchymal Stem Cells” or MSC

The term “Mesenchymal Stem Cell” or “MSC”, as used herein, refers to adult progenitor cells that can self-renew and can differentiate into multiple lineages such as osteoblasts, adipocytes and chondroblasts. MSCs can be isolated from numerous tissues such as bone marrow, adipose tissue, the umbilical cord, liver, muscle, and lung. MSCs adhere to plastic when maintained under standard culture conditions. MSCs express CD73, CD90 and CD105, but under standard culture conditions lack expression of CD31, CD45, CD11b, and CD19 surface molecules.

### Cellular Integrity

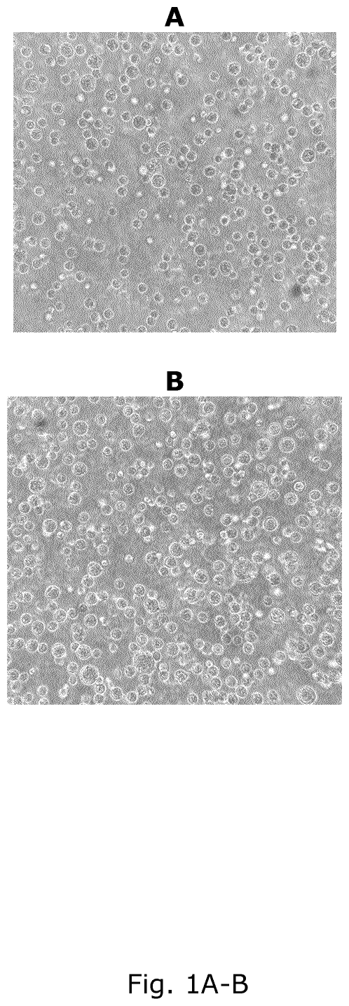

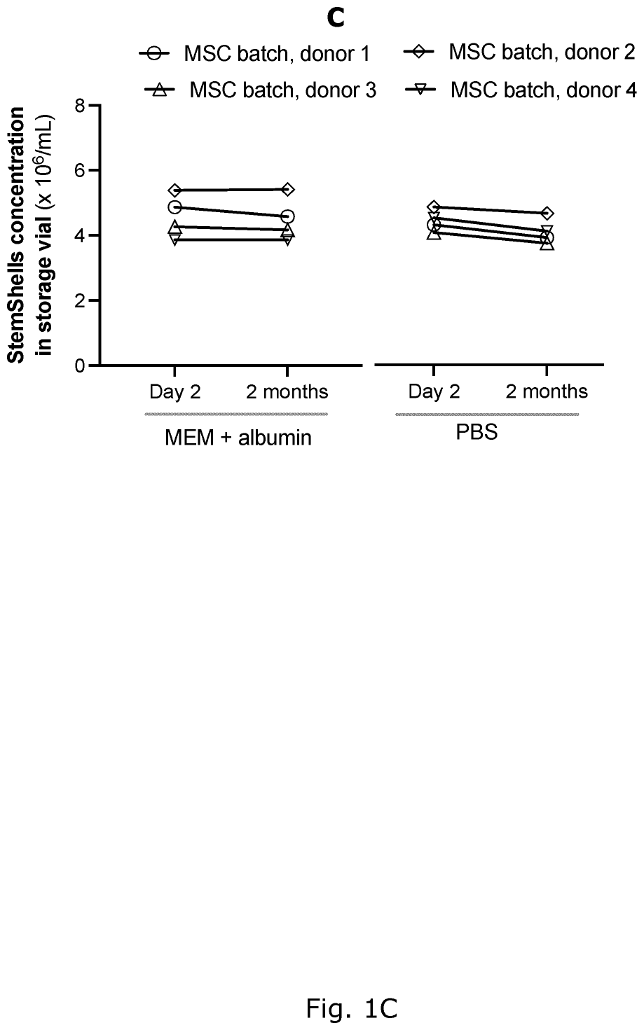

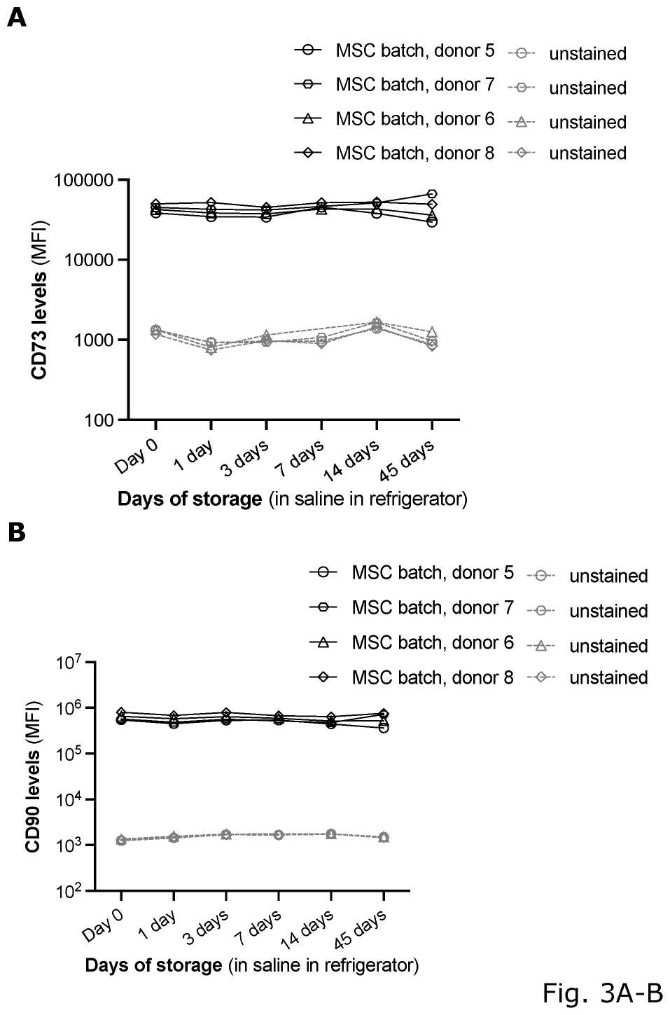

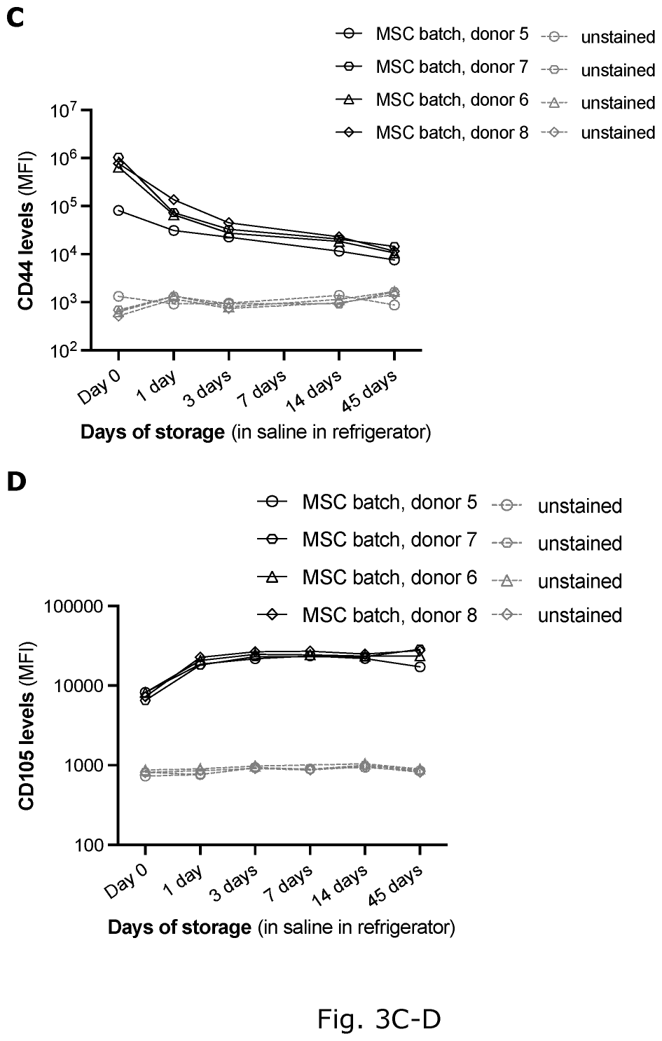

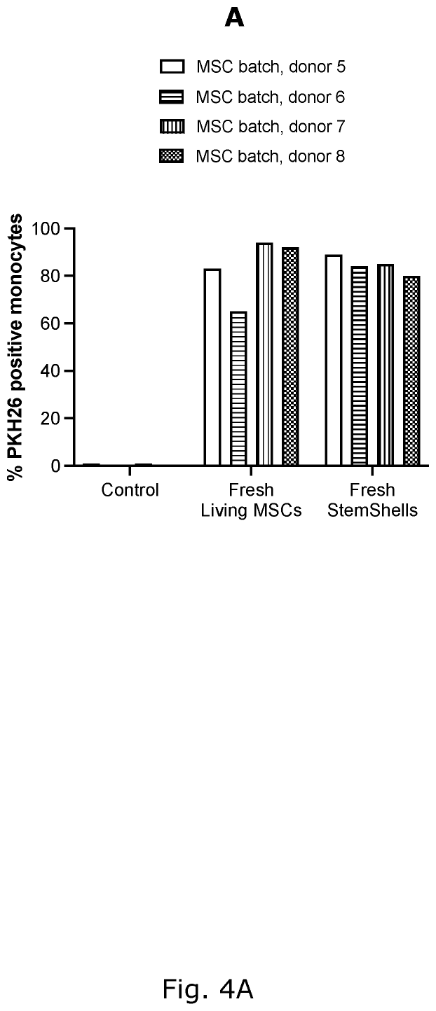

In the present context, the term “cellular integrity” or “maintained their cellular integrity” is to be understood as
i) the metabolically inactivated cells have a clear cellular structure when viewed under a light microscope (FIG. 1A).
ii) the metabolically inactivated cells have the surface marker signature CD73+, CD90+, CD105+, CD44+ (FIG. 3) (see example 4).
iii) The metabolically inactivated cells stain positive for a nuclear staining such as 7-Aminoactinomycin D (7-AAD)

### Balanced Salt Solution (BSS)

In the present context, balanced salt solution (BSS) is a solution made to a physiological pH and isotonic salt concentration. Solutions most commonly include sodium, potassium, calcium, magnesium, and chloride. Examples of balanced salt solutions which may find use with the present invention are:
Isotonic saline;
Alsever's solution;
Earle's balanced salt solution (EBSS);
Gey's balanced salt solution (GBSS);
Hanks' balanced salt solution (HBSS);
(Dulbecco's) Phosphate buffered saline (PBS);
Puck's balanced salt solution;
Ringer's balanced salt solution (RBSS);
Simm's balanced salt solution (SBSS);
TRIS-buffered saline (TBS); and
Tyrode's balanced salt solution (TBSS).

Preferably: Ringer, Saline, Albumin supplemented sodium chloride composition.

Additives may include, antibiotics, vasoconstrictors, growth factor, salt, sugars, or other stimulants.

### ISCT and IFATS Cultured MSC Definition

Mesenchymal and Tissue Stem Cell Committee of the International Society for Cellular Therapy (ISCT) and International Federation for Adipose Therapeutics and Science has the following minimal criteria to define human MSC. First, MSC must be plastic-adherent when maintained in standard culture conditions. Second, cultured MSCs must lack expression of at least 2 negative surface markers, preferably being CD31− and CD45−. In addition, a minimum recommendation is to further characterize the cells by surface markers CD73+ and CD90+. Additionally supplementary characterization is recommended by panel including e.g. CD11b+, CD13+, CD29+, CD44+, CD34+, CD105. Third, MSC must be able to differentiate to osteoblasts, adipocytes and chondroblasts in vitro under the proper stimuli. [8]

### Metabolically Inactivated Cells

In the present context, the terms “metabolically inactivated” refer to cells which are without mitotic and metabolic activity.

In an embodiment, the metabolically inactivated MSCs are metabolically inactivated and cannot reduce MTT to formazan.

In another embodiment, the metabolically inactivated MSCs refers to cells which are secretome deficient. This is defined as the inability to secrete cytokines and growth factors such as vascular endothelial growth factor (VEGF), FGF2, granulocyte colony-stimulating factor (G-CSF), monocyte chemotactic protein-1 (MCP-1), and interleukin (IL)-1Rα, IFN-γ, IL-1β, IL-10, IL-6, and IL-8.

In a further embodiment, the metabolically inactivated MSCs cannot adhere to plastic.

In a further embodiment, the metabolically inactivation of the MSCs is irreversible.

In an embodiment, the metabolically inactivated MSCs are mitotically/lethally/metabolically inactivated.

### Process for Providing Inactivated Mesenchymal Stem Cells (MSCs)

As outlined above, the present invention relates to the discovery that inactivated MSCs can be stored in a refrigerator for long periods of time, while preserving cellular and phenotypic integrity and bioactivity e.g. the ability to immunomodulate monocytes (see example 6). Thus, an aspect of the invention relates to a process for providing inactivated mesenchymal stem cells (MSCs), the process comprising
a) metabolically inactivating MSCs provided from a subject;
b) storing the inactivated cells at a temperature in the range 0.1-10° C., for a period of at least 2 days, such as at least 5 days, such as at least 7 days, such as at least 14 days, such as 45 days; and
c) providing inactivated MSCs, such as inactivated cells being ready for use as a medicament.

In an embodiment, the cells are mitotically/lethally/metabolically inactivated.

In an embodiment, the provided inactivated cells have maintained their cellular integrity, such as maintained their cellular integrity for at least 15 days, such as at least 30 days or such as at least 60 days, when stored in a temperature range between 0.1 and 10° C., such as 1-8° C., such as 2-6° C., such as 3-5° C., or such as around 4° C.

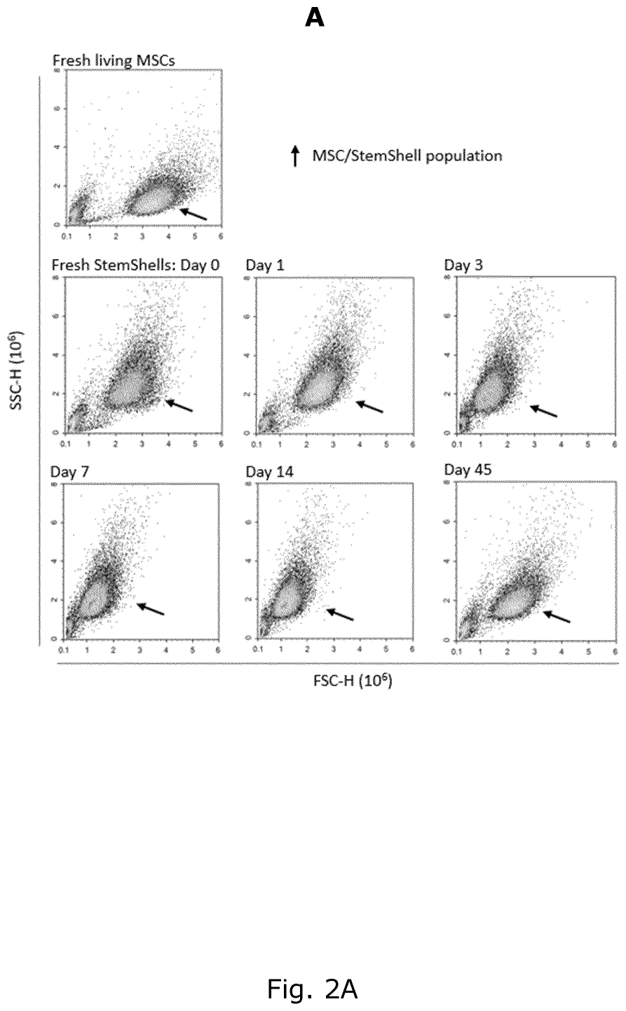

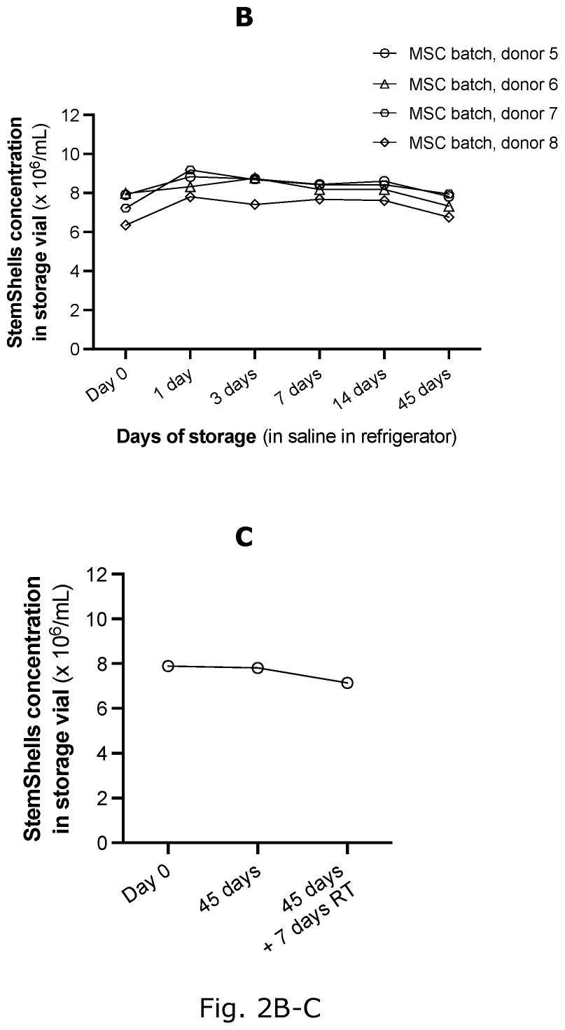

It could also be foreseen to actually store the cells for a period of time at ambient room temperature. Thus, in an embodiment, the provided inactivated cells has maintained their cellular integrity, such as maintained their cellular integrity for at least hours, such as at least 6 hours, such as at least 2 days, such as at least 7 days, such as at least 15 days, such as at least 30 days, such as at least 60 days, when stored in a temperature range 11-28° C., such as 15-28° C., or such as 18-25° C. as seen in FIG. 2C.

In another embodiment, the process is for providing inactivated mesenchymal stem cells (MSCs) suitable for use as a medicament (or for use as a medicament).

### Step a)

MSCs can be inactivated by different means. Thus, in an embodiment, in step a) the MSCs are inactivated by a method selected from the group consisting of heat treatment, radiation such as ultra-violet radiation, ionizing radiation such as X-ray radiation, and/or chemical treatment. The MSCs are preferably inactivated by heat treatment (see example 1).

In yet an embodiment, in step a) the cells are inactivated by heating to a temperature in the range 40-75° C., such as 40-60° C., preferably at 45-55° C., more preferably at about 50° C., for a period from 5 minutes to 2 hours, 10 minutes to 1 hour, preferably such as 15-45 minutes, more preferably for a period of about 30 minutes or such as at least 10 minutes at 45-75° C.

MSCs can be provided from different tissues. Thus, in an embodiment, the MSCs are selected from the group consisting of adipose derived MSCs, human umbilical cord MSCs, bone marrow derived MSCs, dental pulp MSC and induced pluripotent mesenchymal stem cells, preferably the MSCs are adipose derived MSCs.

In a preferred embodiment, the MSCs are adipose derived or bone marrow derived. As shown in e.g. examples 2 and 8, the method according to the invention is applicable to both these types of MSCs, showing that the method is generally applicable to MSCs.

In another embodiment, the adipose derived MSCs are derived from a stromal vascular fraction (SVF).

The MSCs may be modified in different ways before being inactivated. Thus, in an embodiment, the MSCs are primed and/or pretreated MSCs and/or genetically modified MSCs. Examples of priming or pre-treatment are i) incubation with cytokines, interleukins, growth factors such as VEGF or other secreted factors such as damage-associated molecular patterns (DAMPs), ii) pharmacological or chemical agents, iii) exposure to hypoxic conditions, iv) exposure to other cells types such as injured endothelial cells v) expansion of MSCs in 3D conditions.

Thus, in an embodiment, the priming/pretreatment is selected from the group consisting of incubation with factors such as TNFalpa, INFgamma, IL1beta, and damage-associated molecular patterns (DAMPs), incubation with pharmacological or chemical agents, exposure to hypoxic conditions, exposure to other cells types such as injured endothelial cells, and expansion of MSCs in 3D conditions.

In yet an embodiment, the MSCs are derived from a mammal, and preferably a human being.

In a further embodiment, the MSCs are expanded (such as 1-8 passages) or non-expanded MSCs.

In an embodiment, the MSCs (used to produce StemShells) are thawed cryopreserved mesenchymal stem cells or freshly harvested mesenchymal stem cells. As shown in example 9, both options work equally well.

As shown in example 10, inactivation can be performed in different types of liquids (media). Thus, in an embodiment, the MSC are inactivated in a liquids, such as saline or a balanced salt solution (BSS), such as PBS. BSS are considered pharmaceutical acceptable mediums. In yet an embodiment, the media comprises carrier protein such as HSA, such as 0.5%-20% HSA, such as 0.5%-10% HSA, such as 0.5% to 5% HSA, such as 1-3% HAS by weight.

### Step b)

As outlined above the cells can be stored at temperatures above 0° C. Thus, in an embodiment, the cells never reach a temperature below 0° C., such as below −5° C., such as below −15°, such as below −50° C., such as below −80° C., or such as below −120° C., or such as not being cryopreserved.

In another embodiment, in step b) the cells are stored at a temperature in the range 0.1-8° C., such as 0.5-8° C., such as 2-7° C., preferably 2-6° C.

In yet another embodiment, in step b) the cells are stored at a temperature in the range 15-25° C., such as 18-22° C., or such as around 21° C. As also shown in the example section, the cells may also be stores at room temperature, e.g. during transportation. (see e.g. example 3)

In a further embodiment, in step b) the cells are stored
for at least 3 days, such as at least 4 days, such as at least 7 days, such as at least 10 days or such as at least 15 days, such as at least 30 days, such as at least 40 day, such as at least 50 days, such as at least 60 days or such as at least 100 days; or
in the range 2-200 days, such as 2-120 days, such as 2-80 days, such as 3-80 days, such as 3-60 days, 3-50 days, 3-40, 3-30 days, 3-20 days, 3-16 days, 2-10 days, 2-7 days, or 2-5 days, or such as 4-16 days, 7-16 days, or 10-16 days.

As shown in the example section cells stored for up to 60 days have been tested.

Since the cells can be stored in a standard refrigerator, the cells can also be stored in different containers. In an embodiment, in step b) the cells are stored in a cell-delivering device, wherein said cell delivering device is adapted to deliver the cells to a subject in vivo, such as the device being a syringe. By storing the cells in e.g. a syringe the end-user (clinician) could potentially take the syringe from the refrigerator and it will be ready for use.

In an alternative embodiment, in step b) the cells are stored in a container or vial, preferably being different from an incubation flask or cell growth container. Thus, the cells can be store in a container or vial not adapted for cell growth.

The cells can be stored in different media. Thus, in an embodiment, in step b) the cells are stored in a liquid cell medium, such as saline or a balanced salt solution (BSS), such as PBS. BSS are considered pharmaceutical acceptable mediums.

In a further embodiment, the liquid cell medium further comprises sugar, antibiotics, anesthetics, vasoconstrictors, growth factors, cytokines and ions. Such components are known to the skilled person.

In another embodiment, in step b) the cells are stored in a pharmaceutical acceptable composition, such as composition being ready for injection/infusion in a subject. Preferably isotonic saline solution, Ringers Lactate composition, 2-10% albumin solution composition.

The MSCs according to the invention may be inactivated at a different location than the location where the cells may be used as a medicament. The simple storage of the cells according to the invention makes transportation easy. Thus, in an embodiment, the cells are transferred/transported from the site of inactivation to the site where the cells are to be used during step b), such as to be used as a medicament, such as transportation between two different medical units at different locations.

The MSCs may be stored at different concentrations in the medium. Thus, in an embodiment, the cells are stored at a concentration in the range 10,000-100,000,000 cells pr. mL during step b).

Albeit the concentration of the cells may be important, if the cells are going to be directly used as a medicament, which is going to be infused/injected, the numerical value of inactivated MSC may be at least as important as the concentration. Thus, in an embodiment, the cells are stored in a container with a number of cells in the range 10,000-1,000,000,000 during step b), preferably, in the range of 100,000-100,000,000, more preferably in the range of 1,000,000-10,000,000 MSCs.

### Step c)

As outlined in the example section, the cells maintain their cellular integrity after storage. Thus, in an embodiment, in step c) at least 40% of the cells have maintained their cellular integrity, such as at least, such as at least 60%, such as at least 85%, such as at least 90, such as at least 95% of the cells have maintained their cellular integrity, compared to the cells before inactivation.

In another embodiment, in step c) at least 80% of the cells have maintained their cellular integrity, such as at least 85%, such as at least 90, such as at least 95% of the cells have maintained their cellular integrity compared to day 0 after inactivation.

In yet an embodiment, in step c) at least 80% of the metabolically inactivated cells have maintained their cellular integrity, when compared to the cells provided after step a), such as at least 85%, such as at least 90%, or such as 95% of the inactivated cells have maintained their cellular integrity, when compared to the cells provided after step a).

Thus, albeit inactivation (such as heat inactivation) may destroy part of the cells exposed to the inactivation, after inactivation the cells according to the invention are very stable (maintain their cellular integrity).

Cellular integrity can e.g. be determined by identifying the presence of surface marker signature (indicating that the cells are indeed intact), e.g. by flow cytometry or FACS. Thus, in an embodiment, in step c) at least 80% of the metabolically inactivated cells have the cell surface marker signature CD73+, CD90+, such as CD29+, CD44+, CD73+, CD90+ and CD105+, or such as CD31−, CD45−, CD73+, CD90+, or such as CD29+, CD44+, CD73+, CD90+, CD105+, CD31−, and CD45−, such as at least 85%, such as at least 90% or such as 95% of the inactivated cells have the cell surface marker signature. As outlined in examples 4, the cells indeed have this signature after long term storage.

In yet an embodiment, in step c) at least 80% of the metabolically inactivated cells stain positive for 7AAD, such as at least 85%, such as at least 90% of the cells, or such as at least 95%, or at least 99% of the metabolically inactivated cells. 7AAD positive staining indicate that the inactivated cells have a nucleus and therefore also maintain cellular integrity. See example 2

In yet another embodiment, in step c) at least 80% of the mitotically inactivated cells cannot adhere to plastic, such as at least 85%, such as at least 90% of the cells, or such as at least 95%, or at least 99% of the inactivated cells cannot adhere to plastic.

Example of plastic materials used plastic culture vessels includes polyethylene terephthalate (PET), high-and low-density polyethylene (PE), polyvinyl chloride (PVC) and polypropylene (PP), but the material most frequently used in labs today is polystyrene (PS). Plastics may be further modified.

### Metabolically Inactivated Mesenchymal Stem Cells (MSCs) Obtained/Obtainable by Process

The cells which have been stored according to the invention may have unique properties. Thus, an aspect of the invention relates to metabolically inactivated mesenchymal stem cells (MSCs) obtained/obtainable by a process according to the invention.

### Medical Uses

The cells which have been stored according to the invention may be used as medicaments (see example 5). Thus, yet an aspect of the invention relates to the metabolically inactivated mesenchymal stem cells (MSCs) according to the invention for use as a medicament.

Yet an aspect of the invention relates to metabolically inactivated mesenchymal stem cells (MSCs) for use as a medicament,

wherein said MSCs have been stored at a temperature in the range 0.1-10° C., for a period of at least 2 days, such as at least 5 days, such as at least 7 days, such as at least 14 days, such as at least 60 days before use as a medicament. As outlined in the example section, cells stored for up to 45 days have been found to be effective (see example 5 and 6).

MSCs have been found to be effective in the treatment of a wide varieties of diseases. Thus, yet another aspect of the invention relates to metabolically inactivated mesenchymal stem cells (MSCs) according to the invention for use as a medicament for immunomodulatory and regenerative indications. This includes the treatment of inflammatory diseases, such as acute or chronic inflammatory diseases, including the treatment of autoimmune diseases, treatment of tissue reconstruction, tissue regeneration and/or the treatment and prevention of transplant rejection.

In an embodiment, the metabolically inactivated mesenchymal stem cells (MSCs) are for use as the treatment for inflammatory/immunomodulatory indications which can be selected from the group consisting of sepsis, asthma, celiac disease, glomerulonephritis, Crohn's disease, psoriasis, rheumatoid arthritis, multiple sclerosis, vasculitis, lupus erythematosus, graft-versus-host disease, transplant rejection, and for regenerative indications which can be selected from the group consisting of stroke, osteoarthritis, bone healing, soft tissue reconstruction, wound healing, acute myocardial infarction, alopecia.

In a more preferred embodiment, the inflammatory/immunomodulatory/regenerative indication is selected from the group consisting of sepsis, organ transplantation, Crohns disease, rheumatoid arthritis, and diabetic wounds.

In an embodiment the metabolically inactivated mesenchymal stem cells are preferably used for inflammatory diseases as prior art supports such mechanism for freshly mitotically and metabolically inactivated MSCs [7].

The MSCs according to the invention may be used as medicaments in both the subject from who the cells have been obtained (autograft) or in a different subject (e.g. allograft). Thus, in an embodiment, the metabolically inactivated MSCs according to the invention are for use is as an allograft, autograft, isograft or xenograft, preferably an allograft.

The “subject” as described herein is supposed to receive the composition and comprises humans of all ages, other primates (e.g., cynomolgus monkeys, rhesus monkeys); mammals in general, including commercially relevant mammals such as cattle, pigs, horses, sheep, goats, mink, ferrets, hamsters, cats, dogs; and/or birds. Preferred subjects are humans.

Thus, in an embodiment of the present invention, the subject is selected from the group consisting of; humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds.

The MSCs according to the invention can be provided to the subject by different administration routes. Administration of the composition can be done in a number of ways as described in the following, non-limiting, examples. By intradermal injection, which is a delivery into the dermis of the skin, located between epidermis and the dermis. Alternatively, the composition can be administered intravenous, which is an administration directly into the blood stream of the subject. Further, administration of the composition intramuscular is an injection into the muscles of the subject. In addition, the composition can be administered subcutaneous, which is under the skin, in the area between the muscle and the skin of the subject. Further, the composition can be administered intratracheal, which is administration directly into the trachea, transdermal, which is administration across the skin. Intracavity administration includes, but is not limited to administration into oral, vaginal, rectal, nasal, peritoneal, or intestinal cavities as well as, intrathecal. (i.e., into spinal canal), intraventricular (i.e., into the brain ventricles or the heart ventricles), intraatrial (i.e., into the heart atrium) and sub arachnoid (i.e., into the sub arachnoid spaces of the brain) administration. In addition, the composition can be delivered intrasynovial administration which is administrated in the synovium cavity of joints.

Any mode of administration can be used as long as the mode results in the delivery of the composition in the desired tissue, in an amount sufficient to treat the disease.

Preferably, the cells are administered via intravenous infusion, intra dermal injection, intra synovial injection, subcutaneous injection or intramuscular injection. Administration means of the present invention includes; needle injection, catheter infusion, biolistic injections, particle accelerators, needle-free jet injection, osmotic pumps, oral tablets or topical skin cream. Further, Energy assisted plasmid delivery (EAPD) methods or such methods involving the application of brief electrical pulses to injected tissues, commonly known as electroporation may be used to administer the composition according to the invention.

A related aspect of the invention relates to a pharmaceutical composition comprising the metabolically inactivated mesenchymal stem cells (MSCs) according to the invention, for use according to the invention, wherein said pharmaceutical composition comprises in the range 10,000-1,000,000,000 of the metabolically inactivated mesenchymal stem cells (MSCs). Preferably, in the range of 100,000-100,000,000, more preferably in the range of 1,000,000-10,000,000 MSCs.

### Cell Storing or Cell Delivering Device

As outlined above, the cells according to the invention could potentially be stored in a refrigerator in a container or a ready-to-use device. Thus, an aspect of the invention relates to a container or vial comprising the cells according to the invention.

In an embodiment, the container or vial is different from a cell culture vessel for mammalian cells (such as a cell growth container e.g. adapted for attachment of cells to the surface)). Thus, the container or vial may be a container or vial not adapted for cell growth and/or cell adherence.

In yet an embodiment, the cells in the container or vial are in a pharmaceutical acceptable medium.

In yet an embodiment, the container or vial is a cell delivering device, wherein said device is adapted to deliver the cells to a subject in vivo, such as the device being a syringe.

### In Vitro Uses

As outlined above, the cells according to the invention can be used as medicaments in vivo. However, the cells may also find use in vitro. Thus, yet a further aspect of the invention relates to the in vitro use of metabolically inactivated mesenchymal stem cells (MSCs) according to the invention.

In an embodiment, the in vitro use is as a cell stabilizer, a feeder cell, as control in diagnostic assays, or as mean for calibration of medical instruments.

### Other Aspects

Yet another aspect of the invention relates to the use of a cooling container having a storage temperature in the range 0.1-10° C. for storing inactivated MSC until the MSCs are to be used as a medicament.

In an aspect of the invention, the invention relates to a method for treating a subject in need thereof, the method comprising administering to said subject a composition comprising the inactivated and stored MSCs according to the invention to the subject.

In yet an aspect, the invention relates to a method for transporting inactivated mesenchymal stem cells (MSCs), the method comprising transporting the inactivated stem cells from one location to another while keeping the inactivated cells at a temperature in the range 0.1-10° C., e.g. wherein said transportation takes from 2 hours to 20 days, such as 1 day to 20 days such as 2-20 days.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Materials and Methods

### MSC and Blood Donors

The research protocol used was approved by The Central Denmark Region Committees on Health Research Ethics, case number 1-10-72-1-20. The study was performed in accordance with the Helsinki Declaration II.

### Isolation and Culture of Adipose-Tissue Derived Mesenchymal Stem Cells

10 mL aspirated adipose tissue was collected as waste material from 4 female adults undergoing cosmetic surgery. All participants were completely anonymized and no personal data was extracted. The lipoaspirate was processed individually with clinical grade-compliant reagents to produce a stromal vascular fraction (SVF), as previously published by our research group [9]. In brief, the lipoaspirate was washed and processed by enzymatic digestion in combination with mechanically disruption using collagenase (Type IV) (Nordmark Biochemicals-N0002779) and the GentleMACS Octo Dissociator with C-tubes (Miltenyi). The SVF was pipetted into a T175 cultureflask (Greiner bio-one-Gr-660175), for expansion and incubated in incubator at 37° C. humid air and 5% CO₂. The cell culture medium consisted of Minimum Essential Medium (aMEM) (Gibco-Thermo Fisher Scientific-22561-021), 5% PLTGold Human Platelet Lysate (Mill Creek Life Sciences-PLTGOLD100GMP), 1% L-Glutamin (Gibco-Thermo Fisher Scientific-25030-024) and 1% Penicillin-Streptomycin (Gibco-Thermo Fisher Scientific-15070063)). The culture-medium was changed twice during each passage. AD-MSCs at third passage were harvested using 6 mL TrypLE™ Select (Gibco-Thermo Fischer Scientific-A1217701) at 90% confluency, reaching 5-8×10⁶ AD-MSC pr. culture flask. Finally, AD-MSCs from all culture flasks were pooled and aliquoted at 5×10⁶ AD-MSC/tube for cryopreservation using cryoprevervant CryoStor CS10 (Stemcell Technologies-07952). The AD-MSC was stored at −80° C. and used for study tests within 6 months.

### Flow Cytometric Phenotyping of Baseline MSC

Phenotypical characterization was performed as previously published by our research group [10]. For each donor, freshly harvested MSCs at passage 3 was analyzed just before heat inactivation. The cells were analyzed on a Novocyte flow cytometer. The cells were incubated for 30 minutes with antibodies (CD29, CD31, CD34, CD44, CD45, CD73, CD90, and CD105 and as viability-marker, 7AAD) and relevant isotype controls. Fluorescence-minus-one (FMO) controls were used as negative controls for gating strategy.

### StemShell Preparation

For each donor, MSCs were thawed at passage 2 seeded in two T175 flasks and expanded until 80-90% confluency reaching passage 3. The MSCs were trypsinized and reconstituted in 50 degrees Celsius pre-warmed PBS (DPBS+calcium+magnecium) at a concentration of 5×10⁶ AD-MSC/ml. The tubes were transferred to a plated heater and heated at 50° C. for 35 min for heat inactivation. The, now metabolically inactivated MSCs (StemShells), were cooled in ice bath for 5 min and pooled. Next, StemShells were centrifuged at 440×G for 5 min and the supernatant discarded. To reach final formulation, the pellet was divided in 3 portions and resuspended in either PBS (DPBS+Calcium+Magnesium) and MEM medium supplemented with albumin (Advanced-MEM, Thermo Fisher Scientific—12492013) at a concentration of 5×10⁶ StemShells/ml or Saline at a concentration of 10×10⁶ StemShells/ml. The samples were immediately transferred to a refrigerator for long term storage at 4° C.

Inactivation was validated by seeding cells in culture dishes containing cell culture medium at culture conditions, immediately after heat inactivation protocol. After 24 hours, no adherent cells were observed in heat inactivated samples which contrasted to their non-heat inactivated living counterparts (data not shown).

### Stemshell Storage Stability and Viability

StemShells were analyzed at day 0, 1, 3, 7, 11, 14, 16, 30, 45, 60. A StemShell sample was extracted from fridge storage. The StemShells were resuspended and diluted 0.5×10⁶ StemShells/ml using cell culture media. A 100 ul sample was withdrawn for analysis. 1 ul of 7AAD diluted with 4 ul 0.1% BSA/PBS was added to the StemShell sample and incubated at room temperature for 5 min in dark. After incubation, 300 ul of cell culture media was added and the sample was analyzed on a Novocyte flow cytometer for quantification and viability measure.

### Flow Cytometric Phenotyping of StemShells

For each donor, StemShells at day 0, 1, 3, 7, 14 and day 45 were analyzed on a Novocyte flow cytometer. Phenotypical characterization was performed as previously published by our research group [10]. Briefly, the cells were incubated for 30 minutes with antibodies (CD31, CD34, CD44, CD45, CD73, CD90, and CD105 and as viability-marker 7AAD) and relevant isotype controls. Fluorescence-minus-one (FMO) controls were used as negative controls for gating strategy.

### PKH26 Staining of StemShells

PKH26 staining was performed before heat inactivation of MSCs. Briefly, MSCs was stained with PKH26 using 1.3 ul PKH26/10⁶ MSCs using the PKH26 kit (PKH26 Red Fluorescent Cell Linker Kit, Sigma Aldrich). The staining procedure was performed for 60 seconds followed by inactivation of PKH26 by addition of cell culture media. The PKH26 stained MSCs was centrifuged at 440 g for 5 min and washed with cell culture media, this procedure was repeated for a total of four washes to complete the staining protocol. Following staining, the PKH26 positive MSCs were counted by flowcytometry and heat inactivated following previously mentioned protocol.

### Phagocytosis of StemShells by Monocytes.

Human blood samples were obtained from five fully anonymized donors. Flowcytometric analysis of the monocytes in human whole blood was performed by incubation of specific antibodies (CD14, CD16, CD45, CD91 and CD163) for 30 min in 100 ul of whole blood in a propylene tube. Before incubation with antibodies, the blood samples were washed using 0.1% BSA/PBS. After incubation, erythrolysis was performed. Following erythrolysis, samples was washed and analyzed on a novocyte flow cytometer.

A baseline measure of human whole blood was performed by incubating 100 ul of human whole blood at 0 hours with antibodies but without the addition of PKH26 positive StemShells. PKH26 positive StemShells was used for the phagocytosis assay. 50,000 PKH26 positive StemShells was added to 100 ul human whole blood in a propylene tube and incubated in incubator at 5% CO₂ and 37 degrees Celsius. After 3 hours and 24 hours, the PKH26 positive StemShells and whole blood co-culture was incubated with CD14, CD16, CD45 for 30 min and erythrolysis performed before acquiring the flow cytometric data.

### Example 2—Cellular Integrity in Protein Free Solution

### Aim of Study

To determine the cellular integrity of refrigerator stored heat-inactivated MSCs in a protein-rich and a protein-free solution.

### Materials and Methods

Cell types:

Metabolically inactivated mesenchymal adipose tissue-derived stem cells

The StemShells were stored in either PBS or MEM medium supplemented with albumin at a concentration of about 5×10⁶ StemShells/mL. Storage stability was determined at day 0, 1, 3, 7, 14, and 60.

Method of inactivation: See example 1.

Method of determining stability:

Cellular integrity was determined by:
Visual inspection (FIG. 1A-B);
Flow cytometry (FIG. 1C);

### Results

The results shows that StemShells stay structural intact during storage at refrigerator temperatures for at least 60 days, maintaining 4-5×10⁶ StemShells count for every batch, regardless of using a protein-rich buffer or a protein-free solution. StemShells were positive (>95% of all counts) for 7-AAD staining at all analyzed time points and analyzed batches (data now shown). This confirmed the presence of a nucleus.

### Conclusion

These experiments indicate the StemShells can be stored for at least 60 days at 4° C. in a protein-free solution.

### Example 3—Cellular Integrity of Refrigerator Stored StemShells in a Clinically Relevant Isotonic Solution

### Aim of Study

To determine the cellular integrity of refrigerator stored StemShells in a clinically relevant isotonic solution. Further, an aim is to determine if longer exposure at room temperature during e.g. transportation or onsite handling, influence cellular integrity of refrigerator stored StemShells.

### Materials and Methods

Cell types: metabolically inactivated mesenchymal adipose tissue-derived stem cells.

The StemShells were stored in isotonic saline at a concentration of about 10×10⁶ StemShells/mL. Storage stability was determined at day 0, 1, 3, 7, 14, 45

Method of inactivation: See example 1

Method of determining cellular integrity:

Cellular integrity was determined by Flow cytometry.

### Results

The results show that the StemShells are structurally stable for at least 45 days when stored at 4° C. in a clinically relevant and directly injectable solution (isotonic saline) (FIG. 2A-B).

The results also show that 7 days of room temperature exposure after 45 days refrigerator storage still preserves the Cellular integrity of the StemShells (FIG. 2C).

### Conclusion

The results show that StemShells can maintain their cellular integrity for at least 45 days when stored at 2-8° C. in a directly injectable solution. Additionally, the results show that the cellular integrity of the StemShells is preserved after 7 days of storage at room temperature. This is important e.g. in connection with transportation at room temperature.

### Example 4—Phenotypic Integrity of Refrigerator Stored StemShells

### Aim of Study

To determine the phenotypic integrity of refrigerator stored StemShells by characterization of MSC cell surface markers, CD44, CD73, CD90 and CD105.

### Materials and Methods

Cell types: metabolically inactivated mesenchymal adipose tissue-derived stem cells

The StemShells were stored in either isotonic saline at a concentration of about 10×10⁶ StemShells/mL. Phenotypical integrity was determined at day 0, 1, 3, 7, 14, 45.

Method of inactivation: See example 1

Method of determining phenotypic integrity:
Percentage of CD44+, CD73+, CD90+ and CD105+ cells.
Median fluorescent intensity of MSC cell surface markers CD44+, CD73+, CD90+ and CD105+ during storage.

Phenotypic integrity was determined by Flow cytometry.

### Results

The results shows that the level of StemShell surface markers, CD44, CD73, CD90 and CD105 are stable during storage at refrigerator temperatures for at least 45 days (FIG. 3A-D). For all 4 MSC batches, at least 95% of StemShells that were stored in isotonic saline at refrigerator temperatures maintained their MSC phenotype (CD44+, CD73+, CD90+ and CD105+) (data not shown).

The MFI of all cell surface markers were stable during the 45 days of storage, with the exception of CD44.

### Conclusion

The results show that StemShells maintain their phenotypic integrity for at least 45 days when stored at refrigerator temperatures (around 4° C.) in isotonic saline based on cell surface markers.

### Example 5—Monocytic Phagocytosis of 14 Days Stored StemShells

### Aim of Study

To evaluate if StemShells stored in isotonic saline at refrigerator temperatures interact with monocytes similarly to freshly metabolically inactivated MSCs, as presented in prior art.

### Materials and Methods

Cell types: metabolically inactivated mesenchymal adipose tissue-derived stem cells.

StemShells stored in isotonic saline at a concentration of about 10×10⁶ StemShells/mL. Whole blood was incubated with PKH-labeled StemShells or MSCs. After co-incubation phagocytosis by monocytes was determined by the detection of PKH26 positive monocytes.

Method of inactivation: See example 1

Monocytic phagocytosis was determined by Flow cytometry.

### Results

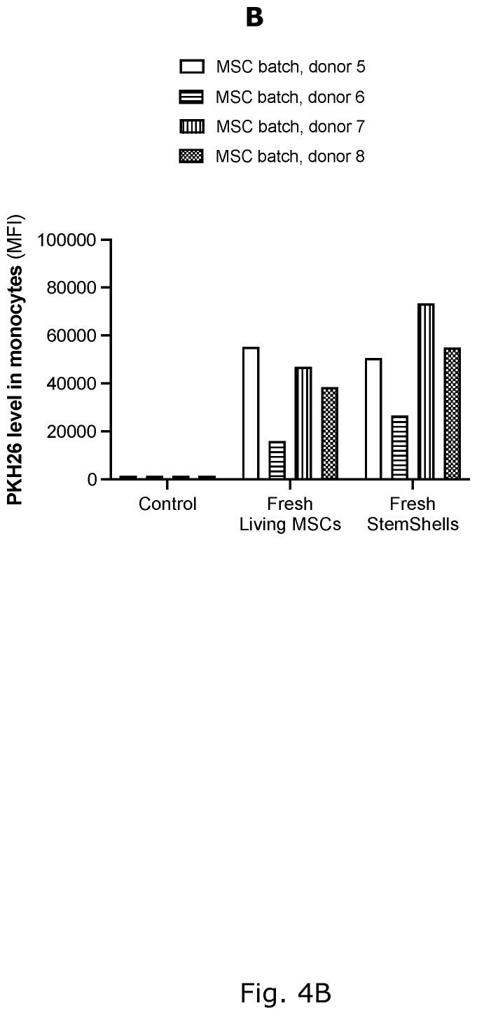

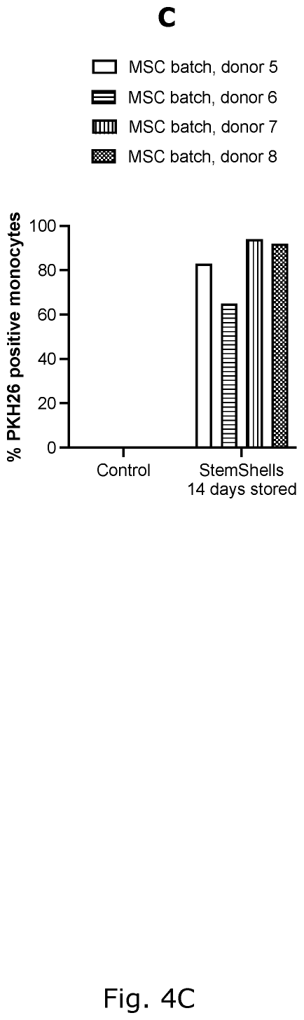

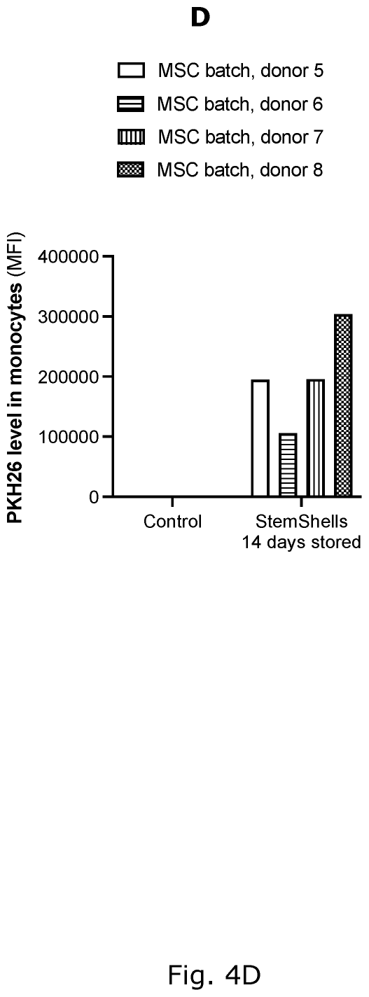

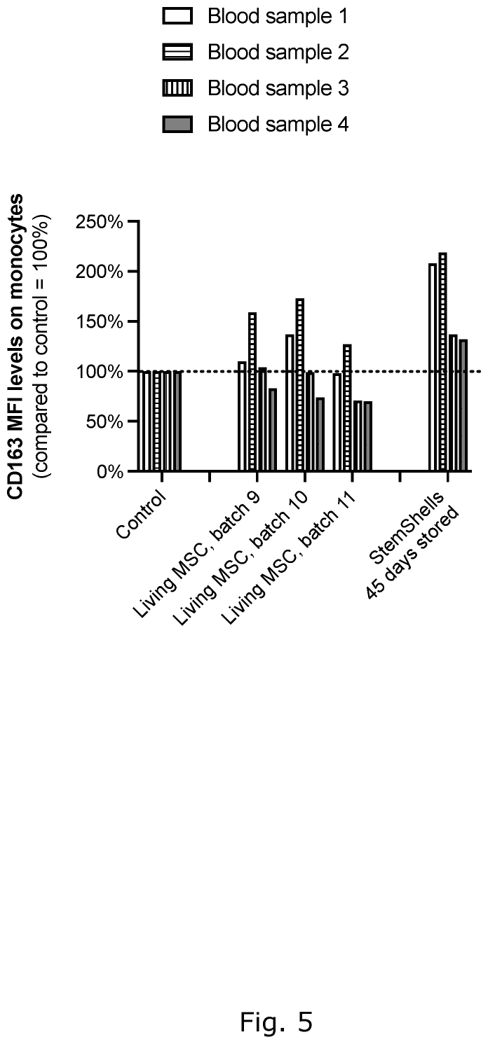

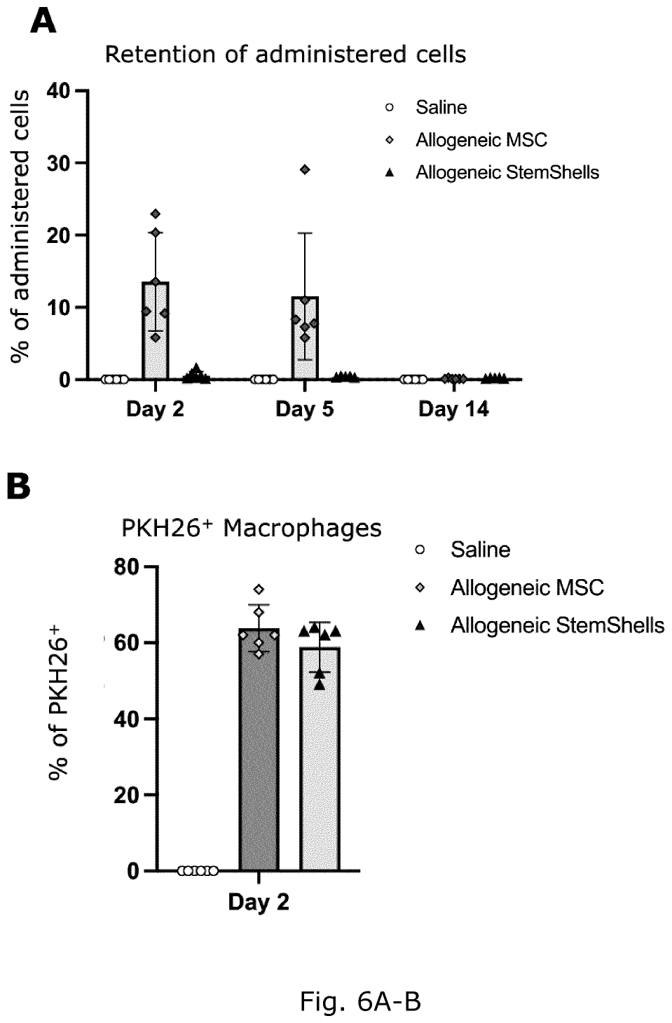

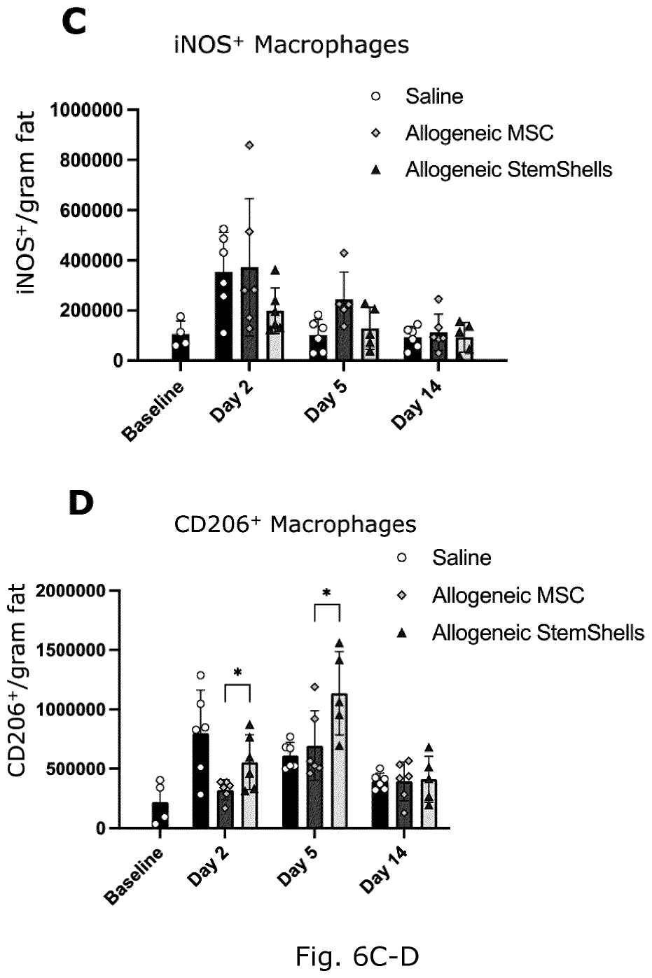

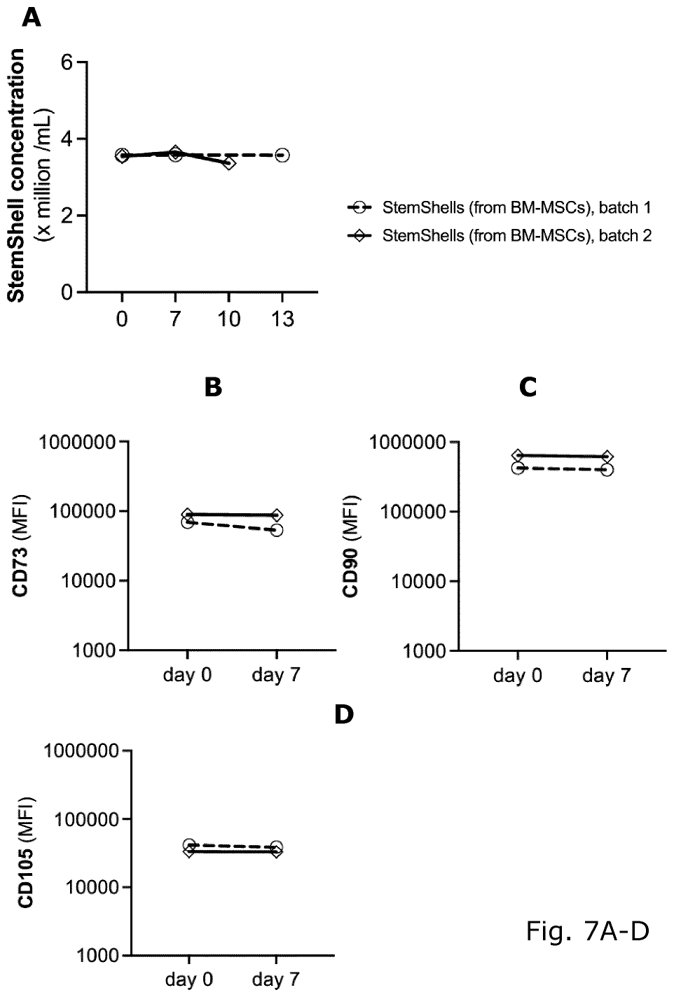

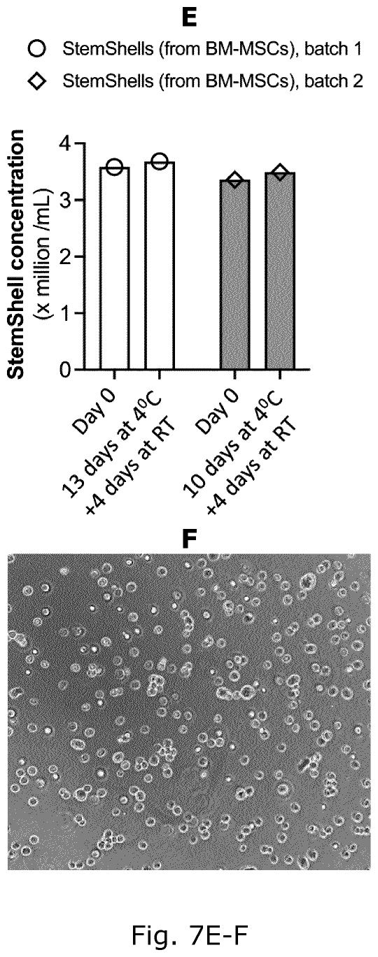

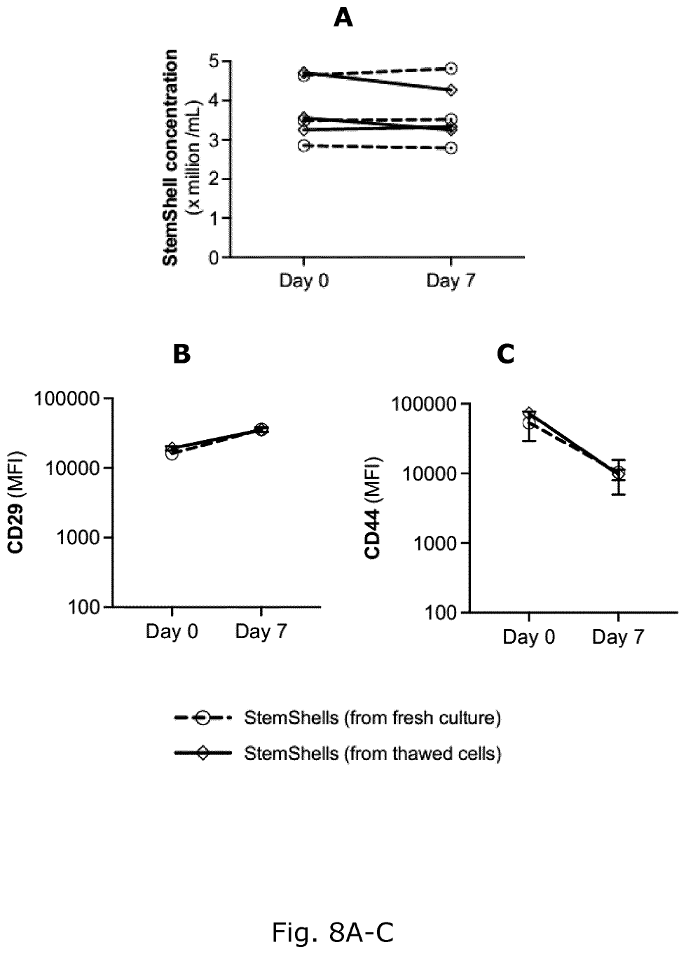

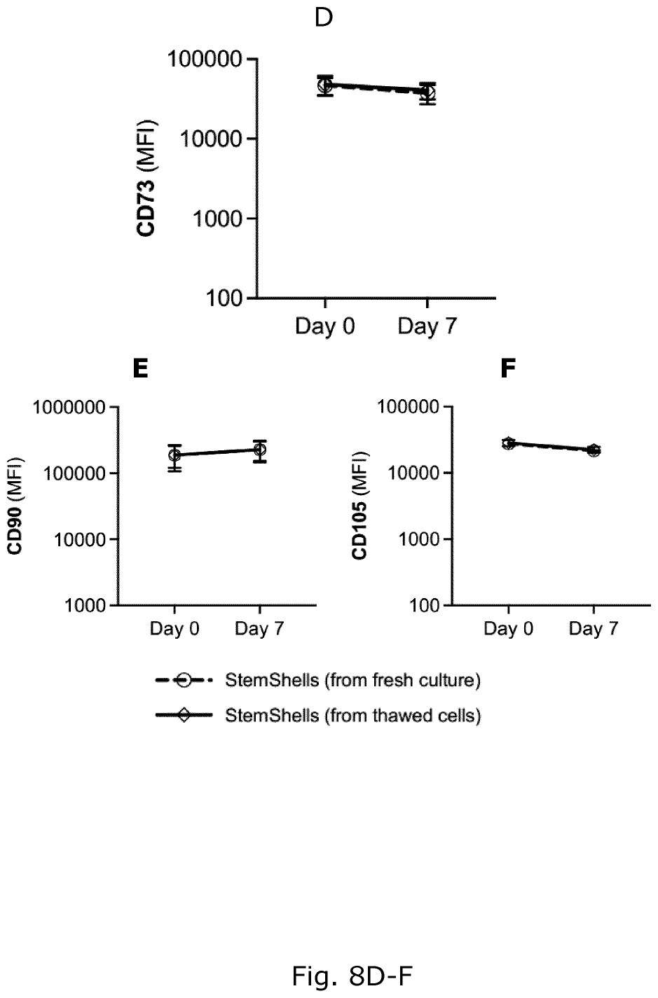

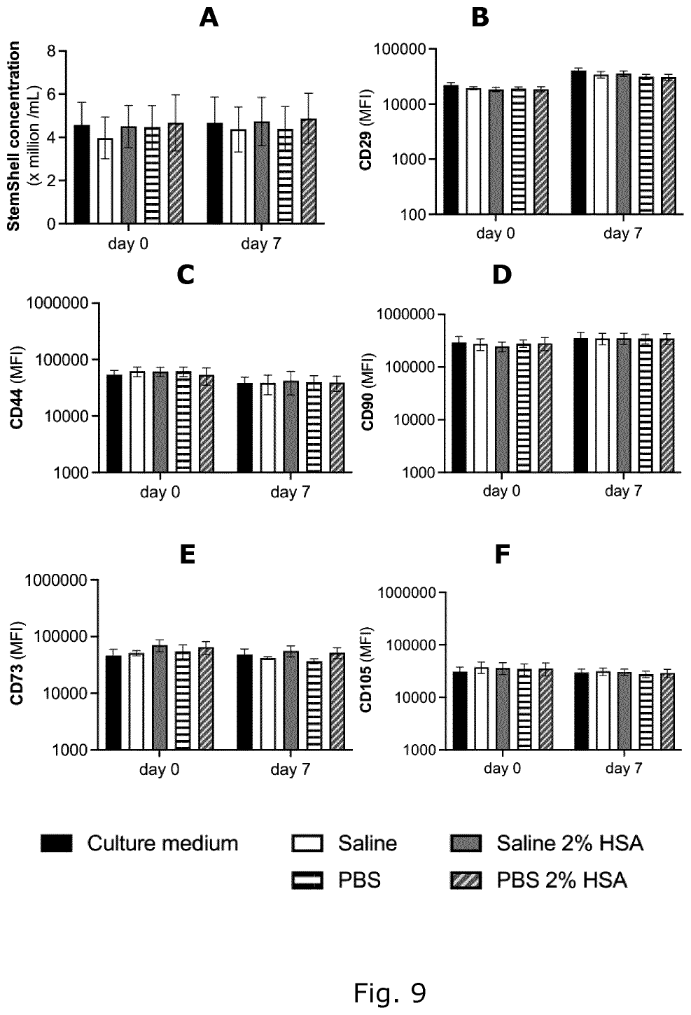

The results show that fresh StemShells (PKH26 labeled) and living MSC (PKH26 labeled) are phagocytized by monocytes as shown in prior art (FIG. 4A-B). Further, StemShells stored in isotonic saline for 14 days at refrigerator temperatures are phagocytized by monocytes in similar manner as both their living MSC counterpart and fresh StemShells (FIG. 4C-D).

### Conclusion

StemShells stored in isotonic saline at refrigerator temperatures for 14 days are efficiently phagocytized by monocytes indicating their immunomodulatory activity.

### Example 6—Immunomodulatory Potency of 45 Days Stored StemShells

### Aim of Study

To evaluate if long term refrigerator stored StemShells are able to induce a phenotypic shift of monocytes towards an immunosuppressive subtype (CD163⁺⁺).

### Materials and Methods

Cell types: metabolically inactivated mesenchymal adipose tissue-derived stem cells.

StemShells stored in isotonic saline at a concentration of about 10×10⁶ Stemshells/mL. Immunomodulatory potency was determined after 45 days of StemShell refrigerator storage.

Method of inactivation: See example 1

Immunomodulatory potency was determined by Flow cytometry.

### Results

When compared to control (whole blood only), StemShells that are stored in isotonic saline for 45 days at refrigerator temperatures induced a CD163 upregulation on CD14+, CD16+ monocytes, which was at least a strong compared to the CD163 upregulation induced by living MSCs (FIG. 5).

### Conclusion

StemShells that are stored in isotonic saline for 45 days at refrigerator temperatures are able induce an immunosuppressive phenotype of monocytes, demonstrating their immunomodulatory therapeutic potential.

### Example 7—In-Vivo Phagocytosis and Immunomodulatory Potency of 7 Days Stored StemShells in Fat Grafts

### Aim of Study

To evaluate if long-term refrigerator stored StemShells are phagocytized by macrophages in-vivo and able to induce a phenotypic shift of these macrophages towards an immunosuppressive subtype (iNOS⁻ and CD206⁺).

### Materials and Methods

Cell types:

Metabolically inactivated mesenchymal adipose tissue-derived stem cells (StemShells) derived from Brown Norway rats. Stem cells was expanded to 3^{rd} passage in culture media consisting of alfa-MEM supplemented with 15% fetal bovine serum, 1% glutamine and 1% penicillin/streptomycin.

Method of inactivation:

See example 1

Experimental model:

Immunomodulatory potency of 7 days refrigerator stored StemShells was determined after 2, 5 and 14 days of transplantation in rat fat grafts. StemShells was stored in isotonic saline at a concentration of about 10×10⁶ StemShells/mL. StemShell tracing and macrophage phagocytosis of StemShells was performed by labeling of the StemShells before transplantation using membrane label PKH26. Autologous rat inguinal adipose tissue was harvested from female Lewis rats and minced for 1 minute to obtain the fat graft. StemShells was then mixed with the fat graft using two syringes connected by a two-way Luer Lock connector. The StemShell supplemented fat graft was then injected to the back of the rats. After either 2, 5 or 14 days of transplantation, the fat grafts were excised and rats euthanized. A single cell solution was made from the harvested fat grafts by collagenase isolation (see example 1). The single cell suspension was analyzed by flow cytometry for StemShell tracing, phagocytosis and immunomodulatory potency.

### Results

StemShells which had been stored in isotonic saline for 7 days at refrigerator temperatures were completely cleared from the fat grafts within 2 days of transplantation (FIG. 6A). This contrasted to their living counterparts (allogeneic MSCs which were not heat inactivated) which remained present for at least 5 days. Macrophages became positive for PKH26 at day 2 indicating active phagocytosis and clearance of the StemShells (FIG. 6B). The number of pro-inflammatory macrophages (iNOS⁺) was reduced at day 2 and 5 in fat grafts supplemented with StemShells when compared to their living counterpart allogeneic MSCs (FIG. 6C). Additionally, the anti-inflammatory subtype of macrophages (CD206⁺) was significantly increased at day 2 and 5 in fat grafts receiving StemShells when compared to their living counterpart allogeneic MSCs (FIG. 6D).

### Conclusion

StemShells stored in isotonic saline for 7 days at refrigerator temperatures are cleared within 2 days when applied locally in-vivo. The StemShells are phagocytized by macrophages in-vivo similar to monocytes in-vitro. StemShells are able induce an immunosuppressive phenotypic shift (INOS⁻ and CD206⁺) of macrophages in-vivo, demonstrating their immunomodulatory therapeutic potential.

### Example 8—Heat Inactivation of Different Types of Mesenchymal Stem Cells

### Aim of Study

To determine if heat inactivation can be performed on different types of mesenchymal stem cells (e.g., MSC from different tissue sources) and to determine if these cells also keep their structural and phenotypic stability during storage at refrigerator temperatures and at room temperature.

### Materials and Methods

Cell types:

Human bone marrow derived mesenchymal stem cells. MSCs were isolated from bone marrow using Lymphoprep™ density gradient medium. The mononuclear fraction was seeded in a culture flask and the adherent fraction was culture expanded (until p3) using culture medium and an expansion protocol similarly as described in example 1 for adipose tissue derived MSCs.

Method of inactivation:

See example 1. Cells were heat inactivated and stored in isotonic saline+2% human serum albumin (saline 2% HSA).

Method of determining cellular integrity:

Cellular integrity was determined by measuring total amount if cells by a Nucleocounter NC-202, and microscopy using 40× magnification

Method of determining phenotypic integrity:
Median fluorescent intensity of MSC cell surface markers CD73+, CD90+ and CD105+ during storage.

Phenotypic integrity was determined by Flow cytometry (see example 1).

### Results

The results show that bone marrow derived StemShells are structurally and phenotypically stable for at least 13 days when stored at 4° C. in a clinically relevant and directly injectable solution (isotonic saline 2% HSA) (FIG. 7A-D). The results also show that exposure of temperatures such as room temperature (RT) are well tolerated by StemShells derived from bone-marrow derived MSCs (FIG. 7E-F).

### Conclusion

The results show that bone marrow derived MSCs can also be stored for longer periods at refrigerator temperatures and at room temperature after they have been inactivated by heat inactivation.

In sum, the method of the invention is considered generally applicable to different types of MSCs.

### Example 9—Heat Inactivation of Cryopreserved Mesenchymal Stem Cells

### Aim of Study

To determine if heat inactivation of thawed cryopreserved mesenchymal stem cells result in equal cell stability during storage at refrigerator temperatures compared to the use of freshly harvested mesenchymal stem cells for heat inactivation.

### Materials and Methods

Cell types:

Freshly harvested and cryopreserved Human adipose tissue derived mesenchymal stem cells. After expansion MSC were resuspended in CryoStor cryopreservation solution (5 million/mL) and cryopreserved at −80° C. After cryopreservation MSC was thawed and used for heat inactivation. Experiment was performed using 3 different batches of MSC (derived from 3 different donor adipose tissue) of both freshly harvested and cryopreserved MSCs.

Method of inactivation:

See example 1. Cells were heat inactivated and stored in isotonic saline+2% human serum albumin (saline 2% HSA).

Method of determining cellular integrity:

Cellular integrity was determined by Flow cytometry.

Method of determining phenotypic integrity:
Median fluorescent intensity of MSC cell surface markers CD29+, CD44+, CD73+, CD90+ and CD105+ during storage.

Phenotypic integrity was determined by Flow cytometry (see example 1).

### Results

The results show that cryopreserved mesenchymal stem cells can be heat inactivated directly after thawing to produce StemShells. Both StemShells produced from freshly harvested MSCs or from thawed cryopreserved MSC are structurally stable for at least 7 days when stored at refrigerator temperatures in a clinically relevant and directly injectable solution (isotonic saline 2% HSA) (FIG. 8A-F). Phenotypic characteristics (CD29, CD44, CD73, CD90, CD105) during these 7 days of storage are also similar between StemShells produced from freshly harvested and StemShells produced from thawed cryopreserved MSCs.

### Conclusion

StemShells can be produced from cryopreserved MSCs directly after thawing of these. StemShells produced from cryopreserved MSCs maintain their cellular and phenotypic integrity for at least 7 days when stored at refrigerator temperatures (around 4° C.) in isotonic saline.

### Example 10—Use of Different Solutions During Heat Inactivation to Generate StemShells

### Aim of Study

To determine the effect of the solutions used during heat inactivation on the cellular and phenotypic integrity during refrigerator storage.

### Materials and Methods

Cell types:

Human adipose tissue derived mesenchymal stem cells. Experiment was performed using three different batches of MSC (derived from three different donor adipose tissue).

Method of inactivation:

See example 1. MSCs were heat inactivated in different solutions (culture medium, saline, saline+2% HSA, PBS, and PBS+2% HSA), washed and reconstituted in isotonic saline 2% HSA for storage.

Method of determining cellular integrity:

Cellular integrity was determined by Flow cytometry.

Method of determining phenotypic integrity:
Median fluorescent intensity of MSC cell surface markers CD29+, CD44+, CD73+, CD90+ and CD105+ during storage.

Phenotypic integrity was determined by Flow cytometry (see example 1)

### Results

The results show that mesenchymal stem cells can be heat inactivated in different solutions, including solutions without protein additives, to produce StemShells which are structurally stable for at least 7 days when stored at 4° C. (FIG. 9A-F). Phenotypic characteristics (CD29, CD44, CD73, CD90 and CD105) was similar between the conditions where StemShells were generated by heat inactivated in different solutions (FIG. 9A-F).

### Conclusion

Heat inactivation to produce StemShells can be performed in protein free solutions such as saline or PBS, solutions supplemented with protein such human serum albumin and complex mixtures such as culture medium.

### Summary of data

The aim of this study was to validate the cellular integrity, phenotypic stability and therapeutic potential of StemShells after long-term storage at refrigerator temperatures in a clinically relevant solution.

In this study series, we have shown that through heat inactivation of MSCs, it is possible to store human adipose tissue-derived mesenchymal stromal cells at refrigerator temperatures for more than 45 days in a clinically relevant solution. The stored StemShells proved structurally stable during the storage (both at refrigerator and room temperature) and maintained the phenotype of their original living counterpart (see examples 2-4).

StemShells stored for 14 days maintained the ability to be phagocytized by human monocytes as seen in prior art (see example 5). Importantly, StemShells stored for 45 days were able to induce a phenotypic shift of monocytes towards an immunosuppressive phenotype in-vitro (see example 6).

Example 7 shows that StemShells are cleared within 2 days when applied locally in-vivo, while still being able to induce an immunosuppressive phenotypic shift (iNOS⁻ and CD206⁺) of macrophages in vivo.

Example 8 shows that bone marrow derived MSCs can also be stored for longer periods at refrigerator temperatures and at room temperature after they have been inactivated by heat inactivation. Thereby documenting that the method of the invention is generally applicable to different types of MSCs.

Example 9 shows that StemShells can be produced from cryopreserved MSCs directly after thawing.

Example 10 shows that heat inactivation to produce StemShells can be performed in protein free solutions such as saline or PBS, solutions supplemented with protein such human serum albumin and complex mixtures such as culture medium.

In conclusion we demonstrated that StemShells (MSC-derived) stored in a clinically relevant solution can be long-term stored at refrigerator temperatures while maintaining their therapeutic potential offering instant clinical access.

### REFERENCES

1. Hoogduijn, M. J. and E. Lombardo, Mesenchymal Stromal Cells Anno 2019: Dawn of the Therapeutic Era? Concise Review. Stem Cells Transl Med, 2019. 8(11): p. 1126-1134.
2. Martin, I., et al., Challenges for mesenchymal stromal cell therapies. Sci Transl Med, 2019. 11(480).
3. Weiss, A. R. R. and M. H. Dahlke, Immunomodulation by Mesenchymal Stem Cells (MSCs): Mechanisms of Action of Living, Apoptotic, and Dead MSCs. Front Immunol, 2019. 10: p. 1191.
4. de Witte, S. F. H., et al., Immunomodulation By Therapeutic Mesenchymal Stromal Cells (MSC) Is Triggered Through Phagocytosis of MSC By Monocytic Cells. Stem Cells, 2018. 36(4): p. 602-615.
5. Weiss, D. J., et al., The Necrobiology of Mesenchymal Stromal Cells Affects Therapeutic Efficacy. Front Immunol, 2019. 10: p. 1228.
6 Luk, F., et al., Inactivated Mesenchymal Stem Cells Maintain Immunomodulatory Capacity. Stem Cells Dev, 2016. 25(18): p. 1342-54.
7. Weiss, A. R. R., et al., Differential effects of heat-inactivated, secretome-deficient MSC and metabolically active MSC in sepsis and allogenic heart transplantation. Stem Cells, 2020.
8. Bourin, P., et al., Stromal cells from the adipose tissue-derived stromal vascular fraction and culture expanded adipose tissue-derived stromal/stem cells: a joint statement of the International Federation for Adipose Therapeutics and Science (IFATS) and the International Society for Cellular Therapy (ISCT). Cytotherapy, 2013. 15(6): p. 641-8.
9. Alstrup, T., et al., Isolation of Adipose Tissue-Derived Stem Cells: Enzymatic Digestion in Combination with Mechanical Distortion to Increase Adipose Tissue-Derived Stem Cell Yield from Human Aspirated Fat. Curr Protoc Stem Cell Biol, 2019. 48(1): p. e68.
10. Alstrup, T., et al., Measured Levels of Human Adipose Tissue-Derived Stem Cells in Adipose Tissue Is Strongly Dependent on Harvesting Method and Stem Cell Isolation Technique. Plastic and reconstructive surgery, 2020. 145(1): p. 142-150.

1. A process for providing metabolically inactivated mesenchymal stem cells (MSCs), the process comprising
a) metabolically inactivating MSCs provided from a subject;
b) storing the inactivated cells at a temperature in the range 0.1-25° C., for a period of at least 2 days; and
c) providing inactivated MSCs.

2. The process according to claim 1, wherein the MSCs are adipose derived or bone marrow derived.

3. The process according to claim 1, wherein the MSCs are adipose derived and derived from a stromal vascular fraction (SVF).

4. The process according claim 1, wherein the cells are lethally and/or metabolically inactivated.

5. The process according to claim 1, wherein the provided inactivated cells in step c) has maintained their cellular integrity, such as maintained their cellular integrity—
for at least 15 days, when stored in a temperature range between 0.1 and 10° C.; and/or
for at least 2 days, when stored in a temperature range 11-28° C.

6. The process according to claim 1, wherein the process is for providing inactivated mesenchymal stem cells (MSCs) suitable for use as a medicament.

7. The process according to claim 1, wherein in step a) the MSCs are inactivated by a method selected from the group consisting of heat treatment, radiation, ultra-violet radiation, ionizing radiation, X-ray radiation, and/or chemical treatment.

8. The process according to claim 1, wherein in step b) the cells are stored in a container or vial.

9. The process according to claim 1, wherein in step b) the cells are stored in a liquid cell medium.

10. The process according to claim 1, wherein the cells are stored at a concentration in the range 10,000-100,000,000 per ml during step b); and/or
wherein the cells are stored in a container with a number of cells in the range 10,000-1,000,000,000 during step b).

11. The process according to claim 1, wherein in step c)
at least 80% of the metabolically inactivated cells have the cell surface marker signature CD73+, CD90+, such as CD29+, CD44+, CD73+, CD90+ and CD105+ or the cell surface marker signature such as-CD31−, CD45−, CD73+, CD90+, or the cell surface marker signature such as-CD29+, CD44+, CD73+, CD90+, CD105+, CD31−, and CD45−; and/or
at least 80% of the metabolically inactivated cells stain positive for 7AAD; and/or
at least 80% of the metabolically inactivated cells cannot adhere to plastic.

12. The process according to claim 1, wherein the MSCs are derived from a mammal.

13. The process according to claim 1, wherein the MSCs are expanded MSCs.

14. The process according to claim 1, wherein the MSCs are derived a human being.

15. The process according to claim 1, wherein the process is for providing inactivated mesenchymal stem cells (MSCs) suitable for in vitro use as a feeder cell.

16. A method for treating a subject in need thereof, the method comprising administering to said subject a composition comprising an inactivated MSC according to claim 1 to the subject.

17. In vitro use of an inactivated MSC according to claim 1.

18. The in vitro use according to claim 17, wherein the in vitro use is as a feeder cell.
